(19) United States
(12) Patent Application Publication
(10) Pub. No.: US 2026/0202528 A1
(43) Pub. Date: Jul. 16, 2026

(54) APERTURE SPACE TRANSFORM-BASED DATA COMPRESSION

(71) Applicant: KONINKLIJKE PHILIPS N.V., EINDHOVEN (NL)

(72) Inventor: Jeff Sherman, Cambridge, MA (US)

(21) Appl. No.: 19/135,215

(22) PCT Filed: Nov. 24, 2023
(86) PCT No.: PCT/EP2023/082955

§ 371 Date: Jun. 3, 2025

Related U.S. Application Data
(60) Provisional application No. 63/430,137, filed on Dec. 5, 2022.

(51) Int. Cl. G01S 7/52; A61B 8/00

(52) U.S. Cl. CPC G01S 7/52034; A61B 8/4488; A61B 8/5207

(57) ABSTRACT

A medical imaging system (100) includes a processing circuit (115) including a memory (151) and an array (305) of transducers. The processing circuit (115) is configured to: receive data sampled by N transducers of the array (305); digitize the data sampled by the N transducers of the array (305) into digitized data; transform the digitized data into transformed digitized data; compress the transformed digitized data into compressed transformed data; and transmit the compressed transformed data.

### BACKGROUND

Ultrasound systems include a digital probe, an ultrasound base and a display. Modern ultrasound systems operate by using the digital probes to transmit sound waves into the body and record the echoes of the sound waves. The ultrasound systems generate large quantities of digital data from spatial sampling by the digital probes. The digital probes use arrays of transducers to spatially sample the echoes. The arrays of transducers may include dozens, hundreds or thousands of sensors which output voluminous amounts of raw digital data from the spatial sampling of the echoes. Analog to digital converters (ADCs) are used to convert the samples measured by the transducer sensors into the raw digital data. The number of channels in a digital probe is dependent on how fast the raw digital data can be read, which is a function of the ADC sampling rate, the number of bits per sample, and the number of available data lanes connecting the digital probe to the ultrasound base. In other words, channel count in digital probes is primarily limited by the data rate achievable on the ultrasound system. Data rates can be increased in several ways, such as by adding more data lanes to transmit the data, increasing the frequency of the ultrasound system, or using more complex encoding methods (e.g., QAM) to encode more bits in a symbol. These methods require extra space, consume significant amounts of power, or add system design complexity, respectively. When the data rate cannot be increased through these or other methods, data compression can be used to reduce the amount of data per channel. Insofar as memory and power are scarce for digital probe applications, data compression algorithms benefit from being computed in real-time with minimal storage elements. Some digital probes compress data by reducing the accuracy of the ADC, applying decimation filters to resample data at a lower sampling rate, or by implementing hardware-based beamformers to sum multiple channels together. Each of these methods may limit the quality of the resultant image and/or the flexibility of the system.

### SUMMARY

According to an aspect of the present disclosure, a medical imaging system includes a processing circuit including a memory and an array of transducers. The processing circuit is configured to: receive data sampled by N transducers of the array; digitize the data sampled by the N transducers of the array into digitized data; transform the digitized data into transformed digitized data; compress the transformed digitized data into compressed transformed data; and transmit the compressed transformed data.

According to another aspect of the present disclosure, a method for communicating data in a medical imaging system includes receiving, by a processing circuit, data sampled by N transducers of an array; digitizing the data sampled by the N transducers of the array into digitized data; transforming the digitized data into transformed digitized data; compressing the transformed digitized data into compressed transformed data; and transmitting the compressed transformed data.

According to another aspect of the present disclosure, a tangible non-transitory computer-readable storage medium stores a computer program. The computer program, when executed by a processor, causes a system to: receive, by a processing circuit, data sampled by N transducers of an array; digitize the data sampled by the N transducers of the array into digitized data; transform the digitized data into transformed digitized data; compress the transformed digitized data into compressed transformed data; and transmit the compressed transformed data.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a system for aperture space transform-based data compression, in accordance with a representative embodiment.

FIG. 2 illustrates a method for aperture space transform-based data compression, in accordance with a representative embodiment.

FIG. 3 illustrates a circuit for aperture space transform-based data compression, in accordance with a representative embodiment.

FIG. 4A illustrates a histogram of all errors shown across points for aperture space transform-based data compression, in accordance with a representative embodiment.

FIG. 4B illustrates a Gaussian fit of errors of less than +/−2.5 least significant bits for aperture space transform-based data compression, in accordance with a representative embodiment.

FIG. 5A illustrates a reconstruction in a region of rapid change in a comparison of original data to decompressed data, in accordance with a representative embodiment.

FIG. 5B illustrates a reconstruction in a region of large magnitude changes in a comparison of original data to decompressed data, in accordance with a representative embodiment.

FIG. 5C illustrates an error in a reconstruction, in accordance with a representative embodiment.

FIG. 6A illustrates an image calculated from original data, in accordance with a representative embodiment.

FIG. 6B illustrates an image generated from 10% of compressed data, in accordance with a representative embodiment.

FIG. 6C illustrates an image generated from 20% of compressed data, in accordance with a representative embodiment.

FIG. 6D illustrates an image generated from 40% of compressed data, in accordance with a representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, a Fourier transform may be taken for the raw digital data from sensors across an ultrasound aperture and may be referred to as an aperture space transform (AST). A spatial frequency transformation on the digital data from samples of echoes across the entire aperture may be used to reduce the data rate necessary to transmit information about all of the channels. The aperture space transform may therefore be used to encode the digital data from the sampled wavefront received along transducer elements. As a result of the transformation, the maximum number of bits needed to encode the digital data from the received wavefront is reduced, allowing for significant power savings in the digital communications circuit of the digital probe. More channels may be simultaneously encoded in a digital probe of an ultrasound system given a fixed data rate. Nevertheless, the teachings herein are not limited to ultrasound systems, and are instead applicable to X-ray and other imaging modes which also use spatial sampling.

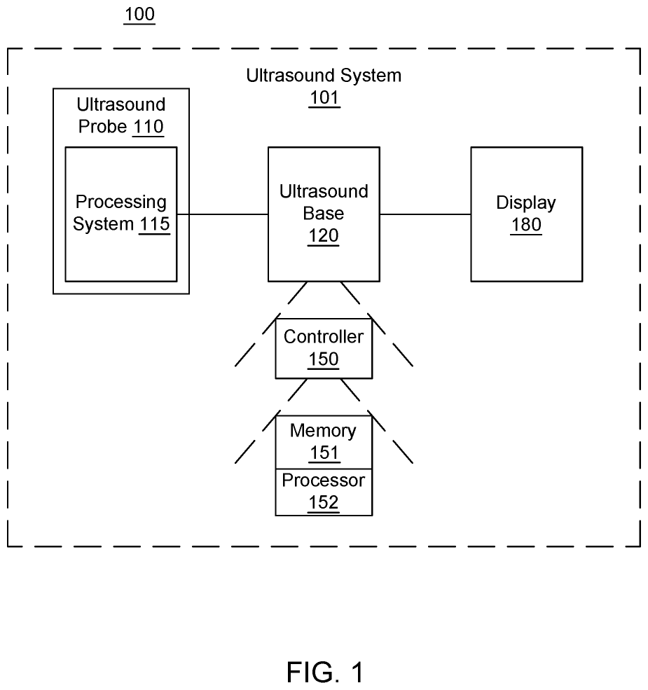

FIG. 1 illustrates a system 100 for aperture space transform-based data compression, in accordance with a representative embodiment.

The system 100 in FIG. 1 is a system for aperture space transform-based data compression and includes components that may be entirely physically connected together or that may be spatially separated and even distributed. The system 100 includes an ultrasound system 101. The ultrasound system 101 includes an ultrasound probe 110, an ultrasound base 120, and a display 180. The ultrasound probe 110 includes a processing system 115. The ultrasound base 120 may be an ultrasound base station and includes a controller 150, and the controller 150 includes a memory 151 and a processor 152.

The ultrasound probe 110 is a digital probe, is mobile, and may be connected to the ultrasound base 120 wirelessly or by wire. The processing system 115 may comprise an array of transducers and a processing circuit. The array of transducers convert electrical energy into sound waves which bounce off of body tissue, and receive echoes of the sound waves and convert the echoes into electrical energy. The array of transducers may include dozens, hundreds or thousands of individual transducer elements. The ultrasound probe 110 may transmit a focus beam to produce high resolution images by sweeping the focus beam and detecting the echoes. Using such a focus beam, only the central elements of the array of transducers will receive a relatively high intensity. Elements of the array of transducers further away from the central elements receive a relatively lower intensity.

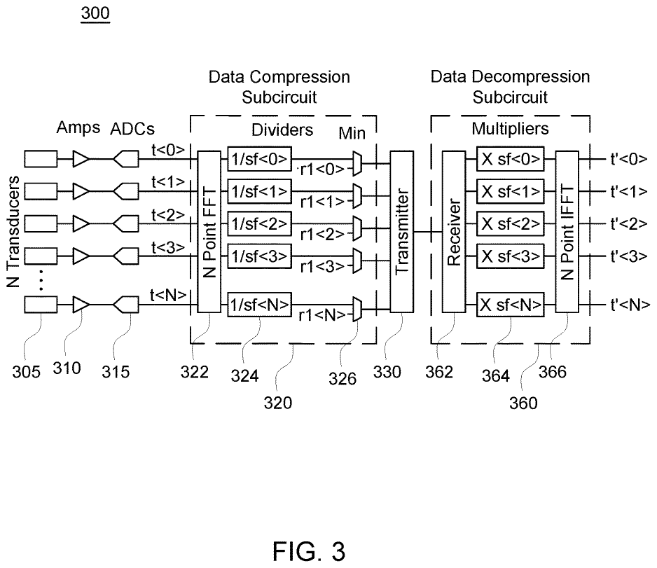

The processing circuit of the processing system 115 may include new digital hardware such as an application-specific integrated circuit (ASIC) to implement a transform such as a fast Fourier transform. An example of an application-specific integrated circuit used to implement the processing circuit of a processing system 115 is shown in and described with respect to FIG. 3. In embodiments in which the processing system 115 includes an application-specific integrated circuit, memory of a processing circuit in the processing system 115 may include registers. In some embodiments, registers in an application-specific integrated circuit serve as memory. Alternatively, the processing circuit may include a controller with a memory that stores instructions and a processor that executes the instructions to perform a software implementation of a transform such as a fast Fourier transform. In some embodiments, memory in the processing circuit may store instructions for execution by a processor. The application-specific integrated circuit or software used to implement the transform in the processing system 115 may be used in any digital transducer probe in the system 100.

Fourier transforms are not traditionally used in processing ultrasound data, at least at the level of the ultrasound probe 110. Fourier transforms may be used to convert data encoded in one domain (such as time) to another domain (such as frequency). Fourier transforms can be used by the processing system 115 to encode spatially sampled data into the frequency domain. A spatial profile when a focus beam is used is relatively easy to encode in a frequency domain (as compared to the time domain) due to the single peak from the focus beam.

The array of transducers of the processing circuit of the processing system 115 may define an aperture. As used herein, an aperture refers to the set of all of the transducer elements of the array of transducers that are currently active. An aperture used for transmission may be different from an aperture used for reception. For example, a transmit aperture may produce narrow focus beams with relatively few aperture elements, whereas the receive aperture may include all of the remaining transducers. As an example of the difference in scale, a transmit aperture may include 16 transducer elements and a receive aperture may include all the transducer elements in an array of 128 or 256 transducer elements.

The ultrasound base 120 may be implemented in any of a variety of forms including as a cart system including a workstation, or as a tablet computer or laptop computer. The controller 150 of the ultrasound base 120 includes at least a memory 151 that stores instructions and a processor 152 that executes the instructions, though a controller 150 may include more elements than depicted in FIG. 1. The memory 151 of the controller 150 of the ultrasound base 120 may store software for implementing an inverse transform such as an inverse fast Fourier transform. The modifications to the ultrasound base 120 to implement the teachings herein may include a new computer program or revisions to an existing computer program stored in the memory 151 and executed by the processor 152. The processor 152 may execute the software to take the transformed data and convert the transformed data back into time data. The time data is then decompressed by a multiplication factor inversely equivalent to the division factors used in the division by the ultrasound probe 110, and the result is the original data.

Memory of a controller described herein may include one or more memories such as a main memory and/or a static memory, where such memories may include instructions executed by a processor and may communicate with each other and other elements of the controller via one or more buses. Memory is a tangible storage medium for storing data and/or executable software instructions, and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory used to store instructions for a controller may be used to implement some or all aspects of methods and processes described herein, along with data used in such methods and processes. The memory used to store instructions may be implemented by any number, type and combination of random access memory (RAM) and read-only memory (ROM), for example. In embodiments in which memories store various types of instructions and information, a processor may cause a controller in the processing system 115 and/or the controller 150 in the ultrasound base to perform various steps and methods using the instructions and information according to the present teachings. Furthermore, updates to the methods and processes described herein may also be stored in such a memory.

The various types ROM and RAM may include any number, type and combination of computer-readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. A computer readable storage medium is defined to be any medium that constitutes patentable subject matter under 35 U.S.C. § 101 and excludes any medium that does not constitute patentable subject matter under 35 U.S.C. § 101. Examples of such media include non-transitory media such as computer memory devices that store information in a format that is readable by a computer or data processing system. More specific examples of non-transitory media include computer disks and non-volatile memories.

The controller 150 and other controllers described herein are representative of one or more processing devices. In embodiments in which controllers comprise memories that store instructions and processors that execute the instructions, the controllers are configured to execute software instructions stored in such memories to perform functions as described in the various embodiments herein. The processor 152 and other processors and processing circuits described herein may be implemented by field programmable gate arrays (FPGAs), systems on a chip (SOC), a central processing unit, a computer processor, a microprocessor, a graphics processing unit (GPU), a microcontroller, a state machine, programmable logic device, or combinations thereof, using any combination of hardware, software, firmware, hard-wired logic circuits, or combinations thereof. Additionally, any processing unit or processor herein may include multiple processors, parallel processors, or both. Multiple processors may be included in, or coupled to, a single device or multiple devices. The term “processor” as used herein encompasses an electronic component able to execute a program or machine executable instruction. References to a device comprising “a processor” should be interpreted to include more than one processor or processing core, as in a multi-core processor.

The display 180 may be local to the ultrasound base 120 or may be remotely connected to the ultrasound base 120. The display 180 may be connected to the ultrasound base 120 via a local wired interface such as an Ethernet cable or via a local wireless interface such as a Wi-Fi connection. The display 180 may be interfaced with other user input devices by which users can input instructions, including mouses, keyboards, thumbwheels and so on. The display 180 may be a monitor such as a computer monitor, a display on a mobile device, an augmented reality display, or another screen configured to display electronic imagery. The display 180 may also include one or more input interface(s) that may connect to other elements or components, as well as an interactive touch screen configured to display prompts to users and collect touch input from users.

The ultrasound probe 110, the ultrasound base 120 and/or the display 180 may also include interfaces, such as a first interface, a second interface, a third interface, and a fourth interface. One or more of the interfaces may include ports, disk drives, wireless antennas, or other types of receiver circuitry that connect the ultrasound probe 110, the ultrasound base 120 and/or the display 180 to other electronic elements. One or more of the interfaces may also include user interfaces such as buttons, keys, a mouse, a microphone, a speaker, a display (separate from the display 180), or other elements that users can use to interact with the ultrasound probe 110, the ultrasound base 120 and/or the display 180 such as to enter instructions and receive output.

The ultrasound probe 110 may perform some of the operations described herein directly and may implement other operations described herein indirectly. For example, the ultrasound probe 110 may indirectly control operations such as by generating and transmitting content to be displayed on the display 180. The ultrasound probe 110 may directly control other operations such as logical operations performed by a processing circuit implemented by the processing system 115. Accordingly, the processes implemented by the ultrasound probe 110 may include steps not directly performed by the ultrasound probe 110.

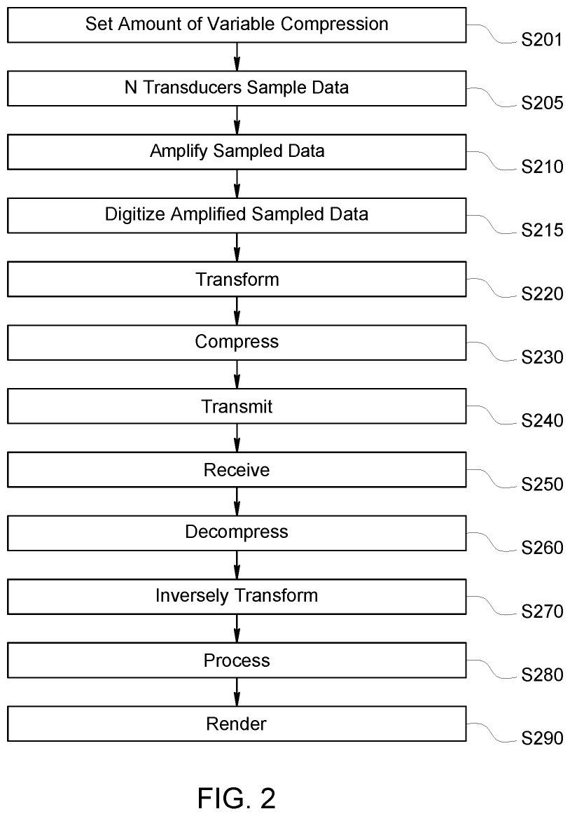

FIG. 2 illustrates a method for aperture space transform-based data compression, in accordance with a representative embodiment.

The method of FIG. 2 may be performed by the ultrasound probe 110 including the processing circuit implemented by the processing system 115. The method of FIG. 2 is based on an understanding of ultrasound waves in the context of a digital transducer where all channels are simultaneously sampled by a transducer array of the processing system 115 and converted into digital signals by the processing circuit of the processing system 115.

At S201, the method of FIG. 2 includes setting an amount of compression. The compression is itself performed at S230, but the amount of variable compression may be set in a variety of manners as described herein, including by varying the scale of division performed by the ultrasound probe 110 on transformed data. The amount of compression may vary as a function of a size of a transmit aperture from the array of transducers, which also corresponds to a width of a transmit beam. The amount of variable compression may be set when the ultrasound probe 110 is built, may be set by an operator of the ultrasound probe 110, or may be set automatically based on other settings set by an operator of the ultrasound probe. Variations of the amount of compression are described later in terms of optimization of the circuit 300 in FIG. 3.

At S205, N transducers of the array of transducers of the processing circuit of the processing system 115 of the ultrasound probe 110 sample data. The data sampled by the N transducers of the array of transducers may be passed to and received by other elements of the processing circuit of the processing system 115. For example, the data sampled by the transducer elements of the array of transducers may be passed to amplifiers.

At S210, the sampled data is amplified. The sampled data is amplified by amplifiers, such as in an application-specific integrated circuit.

At S215, the data sampled by the N transducers of the array and amplified by the amplifiers is digitized into digitized data. The amplified data may be digitized by analog-to-digital converters (ADCs), such as in an application-specific integrated circuit.

At S220, the digitized amplified sampled data is transformed into transformed digitized data. The transformation at S220 may be a Fourier transformation such as a fast Fourier transformation (FFT), and is used to transform spatially sampled data from the aperture space in the time domain into frequency data in the frequency domain. After transmission subsequently at S240, the transformed data may be inversely transformed on the receiving side by software to reproduce the spatially sampled data. In the context of FIG. 1, the receiving side is the ultrasound base 120.

At S230, the transformed data is compressed into compressed transformed data. As noted above, the amount of compression may be variable and set at S201. The processing circuit of the processing system 115 may be configured to be set for an amount of compression as a function of a size of an aperture in the array of transducers. The amount of compression may be varied based on a size of an aperture in the array of transducers. The compression at S230 arises from the relationship between time sampled data on a single channel and the spatially sampled data at a single point in time when imaging is performed using a focused beam. When the focused beam is received at the array of transducers of the processing circuit of the processing system 115, elements in one portion of the array of transducers may receive high voltages, while elements elsewhere in the array of transducers will receive low voltages. Encoding the differences between the high voltages received at some transducer elements and the low voltages received at other transducer elements requires a high dynamic range, and consequently a large number of bits per sample. In comparison, as the spatial beam narrows for high resolution ultrasound applications, the spatial bandwidth broadens, and thus the aperture space representation of a signal is “smeared” across the spectrum of the signal. This “smearing” reduces the necessary dynamic range to encode the signal, reducing the number of bits transferred. The compression may be performed by dividing the digital representation of the transformed data, which effectively may involve deleting or at least ignoring one or more least significant bits to result in compressed transformed data.

At S240, the compressed transformed data is transmitted. The transmission at S240 may be wirelessly or by wire, and may be from the ultrasound probe 110 to the ultrasound base 120 in FIG. 1.

At S250, the transmitted data is received by the ultrasound base 120. The ultrasound base 120 may be an ultrasound base station that receives the compressed transformed data from the ultrasound probe 110.

At S260, the received data is decompressed to obtain the transformed digitized data that was transformed at S230. Decompression may be performed by multiplying the received compressed data, which effectively may involve adding back data for the least significant bits which were cut at S230.

At S270, the decompressed data is inversely transformed to obtain the digitized data that was digitized at S220. That is, the ultrasound base 120 inversely transforms the frequency data back into the time domain to reproduce the spatially sampled data. The inverse transformation at S270 may be an inverse fast Fourier transformation (IFFT), and may be performed by the processor 152 of the controller 150 executing instructions of a software program retrieved from the memory 151 of the controller 150.

At S280, the digitized data is processed. For example, the processing at S280 may be processing to generate, check, filter, or otherwise enhance the digitized data. The spatially sampled data processed at S280 may be usable for generating a display, and the processed data may be transmitted from the ultrasound base 120 to the display 180.

At S290, the processed data is rendered, such as on the display 180.

Using the system 100 of FIG. 1, the ultrasound probe 110 may use a fast Fourier transform as a real-time data compression algorithm in the method of FIG. 2 by transforming the data across the aperture of the ultrasound probe 110, spatially, instead of encoding the data in time. Once the bits of the compressed transformed data are transferred to the ultrasound base 120, the ultrasound base 120 may apply an inverse fast Fourier transform to extract the time trace data with nearly no loss in accuracy. The minimal loss of accuracy is due to cutting only the least significant bit(s) of the transformed data on samples with known low values in the compression. The number of bits needed to transfer data from the ultrasound probe 110 to the ultrasound base 120 is therefore reduced, and this reduction can be used to either save power on the system 100 with fewer channels, or it can be used to allow for more channels to be used in the same power budget. The method of FIG. 2 leverages spatial sampling in the context of how imaging modes such as ultrasound are used. The spatial content is condensed in a way that allows a great reduction of transmitted data compared to when all of raw data is transmitted.

FIG. 3 illustrates a circuit for aperture space transform-based data compression, in accordance with a representative embodiment.

Although termed a “circuit”, the circuit 300 necessarily comprises two or more sub-circuits since the functionally of the circuit 300 is divided between the ultrasound probe 110 and the ultrasound base 120. As shown, the circuit 300 in FIG. 3 includes an array 305 with N transducers, amplifiers 310, ADCs 315, a compression subcircuit 320, a transmitter 330, and a decompression subcircuit 360. The array 305 with N transducers, the amplifiers 310 and the ADCs 315 may form a first application-specific integrated circuit. The compression subcircuit 320 may form a second application-specific integrated circuit. The decompression subcircuit 360 may form a third application-specific integrated circuit. The array 305 with N transducers, the amplifiers 310, the ADCs 315 and the compression subcircuit 320 may be implemented in the ultrasound probe 110. The decompression subcircuit 360 may be implemented in the ultrasound base 120. The ADCs 315 digitize the analog data sampled by the array 305 with N transducers and amplified by the amplifiers 310. The array 305 with N transducers, the amplifiers 310, the ADCs 315 and the compression subcircuit 320 of the circuit 300 may be provided in the ultrasound probe 110 as a modification of the ultrasound probe 110, though in other embodiments, at least some of the functions attributable to these elements may be mostly or entirely performed by software implementations using microcontrollers or field programmable gate arrays (FPGAs).

The compression subcircuit 320 includes logical elements that perform data compression. The logical elements include N transducers of the N point fast Fourier transform 322 which performs an N point fast Fourier transform (FFT), dividers 324, and logic gates 326. The dividers 324 divide digital output created by the N point fast Fourier transform 322 from the digitized data produced by the ADCs 315. The N point fast Fourier transform 322 creates N coefficients, and each of these N coefficients may have a drastically different size due to a smearing effect. The first few coefficients of the output from the N point fast Fourier transform 322 have the highest value, and subsequent coefficients tend to have steadily reduced values with few exceptions. The first coefficients are much larger than the rest due to how the spatial waveform is encoded in the N point fast Fourier transform 322. The reduction by the dividers 324 is performed by dividing down each of these coefficients by a factor, such that the dynamic range of the N point fast Fourier transform 322 is preserved. Multiplication at the decompression subcircuit 360 reverses the division by the compression subcircuit 320.

The division by the dividers 324 may be by a factor of 2, 4, 8, 16, 32 or 64, depending on how many bits of the coefficients from the N point fast Fourier transform 322 may be reasonably eliminated for transmission. Low compression may eliminate 1, 2 or 3 of the least significant bits to divide effectively by 2, 4 or 8, whereas high compression may eliminate 4, 5 or 6 of the least significant bits to divide effectively by 16, 32 or 64. Insofar as output from the ADCs 315 may be 14 bits, each of the N coefficients from the N point fast Fourier transform 322 may start with 14 bits. As a possible limit for the division by the dividers 324, the highest number of bits anticipated at this time to be eligible for division is 7 of 14, so division by 128. The transformed digitized data described herein is compressed by dividing the transformed digitized data to eliminate at least one least significant bit of the transformed digitized data for each of the N transducers, and the compressed transformed data is decompressed by multiplying the compressed transformed data to add back each eliminated bit eliminated when the transformed digitized data is compressed.

Additionally, different coefficients of the N coefficients may be divided by different amounts. For example, if the first five coefficients of the N coefficients are divided by 4 by eliminating the last 2 least significant bits for each coefficient, subsequent coefficients of the N coefficients may be divided by increasingly more. When the division varies in this manner, the last three of the N coefficients may be divided by 64 or 128 by eliminating the last 6 or 7 least significant bits. Division in this manner results in a relatively minute reduction in image quality. In some embodiments using compression as described herein, the data of the N coefficients may be compressed in the order of 20% or 40% with minimal reduction in image quality.

The decompression subcircuit 360 includes a receiver 362, multipliers 364 and a processor 366 which performs an N point inverse fast Fourier transform (IFFT). The decompression circuit obtains the digitized data.

In FIG. 3, the circuit 300 is shown as a block diagram of an implementation of the ultrasound probe 110 and the ultrasound base 120 in FIG. 1. The data sampled across the array 305 with N transducers is notated as the vector t and acts as an input to the N point fast Fourier transform 322. After the compression by the compression subcircuit 320, the encoded information is transmitted across a digital communication channel between the transmitter 330 and the receiver 362. That is, the raw data generated by the sampling by the N transducers is substantially reduced by the compression subcircuit 320 before the transmitter 330 transmits the encoded data. The transformation by the N point fast Fourier transform 322 combined with the compression by the dividers 324 allows for a substantial reduction in the data transmitted by the transmitter 330. Moreover, the compressed data retains the vast majority of the detail from the underlying raw data so that when the compressed data is decompressed by the decompression subcircuit 360, the resultant ultrasound imagery is reliable.

An inverse set of operations decompresses the information at the ultrasound base 120, notated by the vector t′. The digital communication channel in FIG. 3 may be implemented as a cable between the compression subcircuit 320 of the ultrasound probe 110 and the decompression subcircuit 360 of the ultrasound base 120.

Properly choosing the parameters of the fast Fourier transform allows for 1 least significant bit (LSB) of variation, consistent with the noise level of circuit 300, while still allowing for significant bit reduction. The compression in the circuit 300 may be optimized to a desired accuracy. Due to the bitwise complex integer math in hardware, rounding errors may nevertheless occur and cause the compression to be lossy in the circuit 300.

As a first example of optimization in the circuit 300, the implementation of the fast Fourier transform may take the transform of a real valued signal insofar as the fast Fourier transform itself does not assume that input values are real numbers. Because ultrasound probe 110 is sampling the echoes using the array of transducers, the fast Fourier transform used by the ultrasound probe 110 does not assume that any input values are imaginary. As such, only the first half of the transform needs to be computed and transmitted; the second half can be calculated from the first half as the complex conjugate. To encode the fast Fourier transform, only the first half of the fast Fourier transform needs to be encoded since the other half is simply an inverted version. In other words, the symmetry of the fast Fourier transform allows the fast Fourier transform implementation to be optimized to decrease the number of bits being transmitted, and allows for a smaller, more compact hardware implementation or a faster, more power-efficient software implementation. This implementation may also take advantage of symmetries in the algorithm to minimize the number of complex multiplications or reduce the number of bits in an intermediate operation. The number of complex multiplications can be minimized insofar as multiplying by −1 can be computed with a sign bit change, and the latter half of a real fast Fourier transform is the conjugate of the first half. The number of bits in an intermediate operation may be optimized such as when multiplying a branch by one half or one quarter or a constant close to one half or one quarter.

As another example of optimization in the circuit 300, the magnitude of the fast Fourier transform may scale with the length of the transform. On systems with arbitrary resolutions, the transform may be divided by its length so that the spectrum is scaled appropriately. An arbitrary scale factor may be used, such that higher scale factors will reduce the number of bits further, but the higher scale factors will also reduce the signal level on the inverse transform. This factor can be increased until the tolerated minimum signal to noise ratio of the system is met. In addition, the scale factor may be different for every element of the array of N transducers. For example, the first elements in the fast Fourier transform will be the largest, and thus the first elements can be divided down more than other elements without sacrificing the resulting image quality upon the inverse transform. The scale factor is represented in FIG. 3 by the divider blocks and the notation sf<N>.

As another example of optimization in the circuit 300, the maximum magnitude of both the real and imaginary parts of the fast Fourier transform may be shaped by the incoming wave. To take advantage of the relaxed dynamic range on each entry of the fast Fourier transform, each data point in the fast Fourier transform can have a different number of associated bits. For example, the first data point in the fast Fourier transform is completely real-no imaginary data is needed, and so the total number of bits needed to encode the first data point is half of what would be needed to encode the second data point, which has a similar magnitude but a complex representation. As another example: the last set of data points have smaller magnitudes due to the spatial frequency roll-off of the incoming wave, so these elements can be encoded using fewer bits. The resolution limit array may be changed to fit the application to the desired reconstruction accuracy. The resolution limit is represented in FIG. 3 by the min blocks and the notation rl<N>. For samples that rollover the resolution limit, the maximum number can be used as an approximation. Using the maximum number as an approximation may be lossy but shows minimal impact on the circuit 300 if properly set. Stated differently, the scale factor optimization may involve dividing the scale factor down, whereas resolution limit array optimization may involve reducing the total number of bits being transmitted even after the raw digital data is divided.

Other variations may include using multiple smaller transforms across a partial aperture or several partial apertures transformed simultaneously. For very large apertures, such as arrays of transducers with 5000 transducer elements, only a fraction of the transducer elements may optionally be used to avoid having to process too much data. The frame rate may be maintained while using different digital data from shifting subsets of transducer elements, so that each actual measurement is only a part of the total initial sampling data sampled by the transducer array.

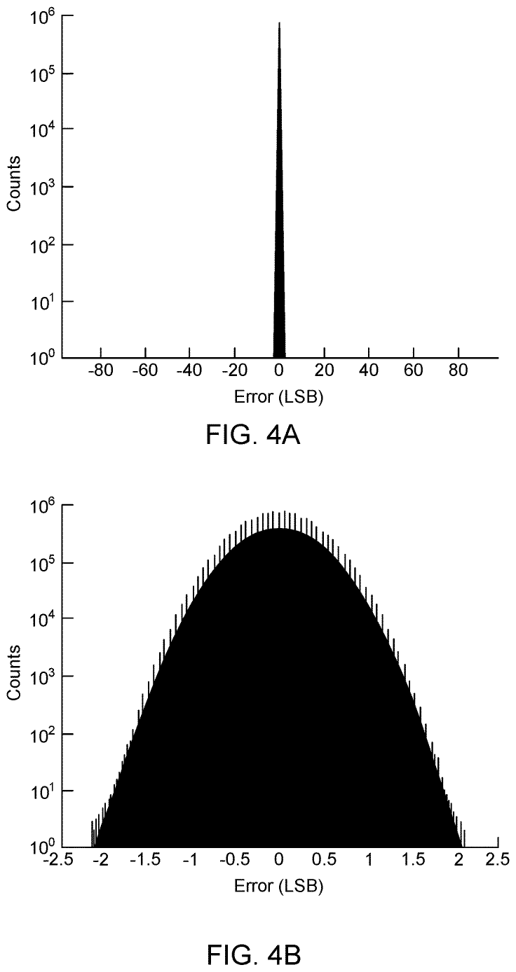

In some embodiments, different representations of complex numbers can be used, such as real and imaginary, magnitude and angle, etc. A simulation of the circuit 300 in practice shows an example of the compression algorithm used across a full aperture. The original phantom data may be sampled across 128 transducer elements with 14 bits of precision. Each horizontal line of data requires 1792 bits to be transferred. In this example, a 128-element fast Fourier transform is performed. The first 65 elements of the transform are compressed using a scale factor array of sf<0:9>=16, sf<10:64>=8, and the resolution limit array used is rl<0:23>=213, rl<24:64>=212. This compression reduces the data to 1593 bits, saving nearly 11% in the bit transfer and power reduction. In this example, the errors follow a Gaussian distribution. The mean error measured μ=6.32×10\hat{ }(−4) LSB, and the standard deviation σ=0.41 LSB across the 199 million reconstructed data points. A histogram of the errors is shown in FIG. 4A, and the Gaussian distribution is shown in FIG. 4B.

FIG. 4A illustrates a histogram of all errors shown across points for aperture space transform-based data compression, in accordance with a representative embodiment. FIG. 4B illustrates a Gaussian fit of errors of less than +/−2.5 least significant bits for aperture space transform-based data compression, in accordance with a representative embodiment.

FIG. 4A and FIG. 4B show a histograms of the error in the compression algorithm. In FIG. 4A, all errors are shown across 199 million points. Errors greater than 2 least significant bits become sparse and rare. In FIG. 4B, errors of less than +/−2.5 least significant bits show a Gaussian fit.

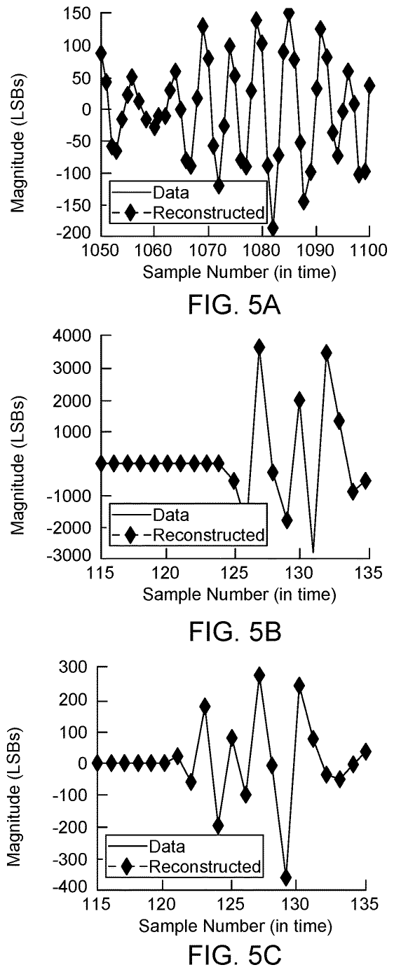

FIG. 5A illustrates a reconstruction in a region of rapid change in a comparison of original data to decompressed data, in accordance with a representative embodiment. FIG. 5B illustrates a reconstruction in a region of large magnitude changes in a comparison of original data to decompressed data, in accordance with a representative embodiment. FIG. 5C illustrates an error in a reconstruction, in accordance with a representative embodiment.

Three example comparisons between the original data and the compressed/decompressed can be seen in FIG. 5A, FIG. 5B and FIG. 5C. FIG. 5A shows close tracking with an area of rapid changes, FIG. 5B shows close tracking with an area of large amplitudes. FIG. 5C shows an area with the largest errors. Collectively, FIG. 5A, FIG. 5B and FIG. 5C illustrate a comparison of original data to decompressed data.

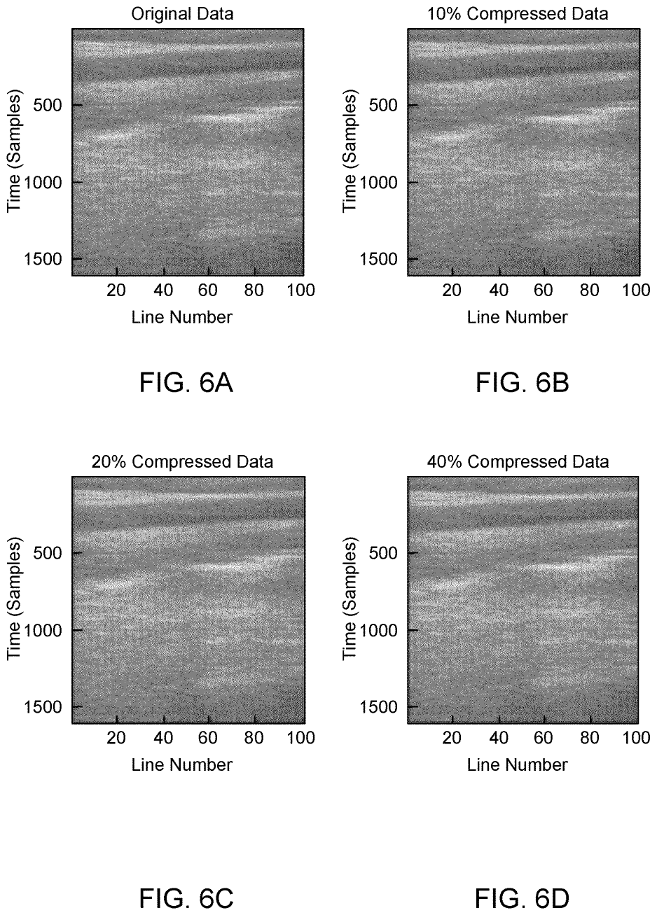

FIG. 6A illustrates an image calculated from original data, in accordance with a representative embodiment. FIG. 6B illustrates an image generated from 10% of compressed data, in accordance with a representative embodiment. FIG. 6C illustrates an image generated from 20% of compressed data, in accordance with a representative embodiment. FIG. 6D illustrates an image generated from 40% of compressed data, in accordance with a representative embodiment.

Images reconstructed from the original data and compressed data sets can be seen in the 2D grayscale plots in FIG. 6A, FIG. 6B, FIG. 6C and FIG. 6D showing different compression ratios and correspondingly higher error rates). FIG. 6A shows an image calculated from the original data. FIG. 6B shows an image calculated from 10% compressed data. FIG. 6C shows an image calculated from 20% compressed data. FIG. 6D shows an image calculated from 40% compressed data. FIG. 6A, FIG. 6B, FIG. 6C and FIG. 6D collecting show reconstructed images using the fast Fourier transform as compression and an inverse fast Fourier transform as decompression.

The control sample in FIG. 6A has no data processing applied, and the three subsequent examples are seen with different settings showcasing results for 10% compression in FIGS. 6B, 20% compression in FIG. 6C and 40% compression in FIG. 6D. The 10% compression in FIG. 6B corresponds to a mean error of μ=6.32×10\hat{ }(−4) LSB and a standard deviation error σ=0.41 LSB). The compression in FIG. 6C corresponds to a mean error of μ=7.9156×10\hat{ }(−4) and a standard deviation error of σ=0.9625. The 40% compression in FIG. 6D corresponds to a mean error of μ=1.31×10\hat{ }(−2) and a standard deviation error of σ=9.6219 LSB.

In some alternative embodiments, a fast Fourier transform may be replaced by applying a wavelet transform across the aperture. The subsequent compression would still be applied insofar as spatial frequency encoding may have a smaller dynamic range than time encoding. In these embodiments, the wavelet transform applied across the aperture may determine an initial set of coefficients, and then the coefficients may be pruned for compression. Since the incoming wavefront is not periodic across the aperture due to the narrowing of the field, a wavelet may encode the wavefront with few coefficients. These embodiments may use a fast Fourier transform to handle the convolution required for the wavelet transform.

The transforms described herein are designed to function on a broadband spatial waveform, and may reduce or eliminate artefacts in the reconstruction. The teachings herein may be applied to any future digital ultrasound probe, and also to any device that uses spatially sampled data with a broadband spatial wavefront. The teachings herein may be used, for example, for neural recording devices with spikey, broadband action potentials localized in an area, and are potentially useful for digital X-ray detection hardware

In an embodiment, dedicated hardware implementations, such as application-specific integrated circuits, field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, a processing circuit may be used to implement a fast Fourier transform on the ultrasound probe 110, in the system 100. An aperture space transform may be taken across an ultrasound aperture of the ultrasound probe 110 and used to encode the wavefront received along transducer elements. As a result of the aperture space transform, the maximum number of bits needed to encode the received wavefront data is reduced, allowing for significant power savings in the digital communications circuit.

Although aperture space transform-based data compression has been described with reference to several exemplary embodiments, it is understood that the words that have been used are words of description and illustration, rather than words of limitation. Changes may be made within the purview of the appended claims, as presently stated and as amended, without departing from the scope and spirit of aperture space transform-based data compression in its aspects. Although aperture space transform-based data compression has been described with reference to particular means, materials and embodiments, aperture space transform-based data compression is not intended to be limited to the particulars disclosed; rather aperture space transform-based data compression extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A medical imaging system, comprising:
a processing circuit including a memory and an array of transducers, wherein the processing circuit is configured to:
receive data sampled by N transducers of the array of transducers;
digitize the data sampled by the N transducers of the array into digitized data;
transform the digitized data into transformed digitized data;
compress the transformed digitized data into compressed transformed data; and
transmit the compressed transformed data.

2. The medical imaging system of claim 1, wherein:
the processing circuit further comprises a processor; and
the memory stores instructions executed by the processor.

3. The medical imaging system of claim 1, wherein:
the processing circuit further comprises an application-specific integrated circuit; and
the memory comprises registers.

4. The medical imaging system of claim 1, further comprising:
an ultrasound base station; and
an ultrasound probe comprising the processing circuit.

5. The medical imaging system of claim 4, wherein the ultrasound base station is configured to:
receive the compressed transformed data;
decompress the compressed transformed data to obtain the transformed digitized data; and
inversely transform the transformed digitized data to obtain the digitized data.

6. The medical imaging system of claim 1,
wherein the transformed digitized data is compressed by dividing the transformed digitized data to eliminate at least one least significant bit of the transformed digitized data for each of the N transducers.

7. The medical imaging system of claim 5,
wherein the transformed digitized data is compressed by dividing the transformed digitized data to eliminate at least one least significant bit of the transformed digitized data for each of the N transducers; and
wherein the compressed transformed data is decompressed by multiplying the compressed transformed data to add back each eliminated bit eliminated when the transformed digitized data is compressed.

8. The medical imaging system of claim 1,
wherein the processing circuit is configured to be set for an amount of compression as a function of a size of an aperture in the array of transducers.

9. The medical imaging system of claim 1, wherein an amount of compression is varied based on a size of an aperture in the array of transducers.

10. A method for communicating data in a medical imaging system, comprising:
receiving, by a processing circuit, data sampled by N transducers of an array;
digitizing the data sampled by the N transducers of the array into digitized data;
transforming the digitized data into transformed digitized data;
compressing the transformed digitized data into compressed transformed data; and
transmitting the compressed transformed data.

11. The method of claim 10, further comprising:
receiving the compressed transformed data;
decompressing the compressed transformed data to obtain the transformed digitized data; and
inversely transforming the transformed digitized data to obtain the digitized data.

12. The method of claim 10,
wherein the transformed digitized data is compressed by dividing the transformed digitized data to eliminate at least one least significant bit of the transformed digitized data for each of the N transducers.

13. The method of claim 11,
wherein the transformed digitized data is compressed by dividing the transformed digitized data to eliminate at least one least significant bit of the transformed digitized data for each of the N transducers; and
wherein the compressed transformed data is decompressed by multiplying the compressed transformed data to add back each eliminated bit eliminated when the transformed digitized data is compressed.

14. The method of claim 10, further comprising:
setting an amount of compression as a function of a size of an aperture in the N transducers.

15. The method of claim 10, wherein an amount of compression is varied based on a size of an aperture in the N transducers.

16. A tangible non-transitory computer-readable storage medium that stores a computer program, wherein the computer program, when executed by a processor, causes a system to:
receive, by a processing circuit, data sampled by N transducers of an array;
digitize the data sampled by the N transducers of the array into digitized data;
transform the digitized data into transformed digitized data;
compress the transformed digitized data into compressed transformed data; and
transmit the compressed transformed data.

17. The tangible non-transitory computer-readable storage medium of claim 16, wherein, when executed by the processor the computer program further causes the system to:
receive the compressed transformed data;
decompress the compressed transformed data to obtain the transformed digitized data; and
inversely transform the transformed digitized data to obtain the digitized data.

18. The tangible non-transitory computer-readable storage medium of claim 16,
wherein the transformed digitized data is compressed by dividing the transformed digitized data to eliminate at least one least significant bit of the transformed digitized data for each of the N transducers.

19. The tangible non-transitory computer-readable storage medium of claim 17,
wherein the transformed digitized data is compressed by dividing the transformed digitized data to eliminate at least one least significant bit of the transformed digitized data for each of the N transducers; and
wherein the compressed transformed data is decompressed by multiplying the compressed transformed data to add back each eliminated bit eliminated when the transformed digitized data is compressed.
